(19) United States
(12) Patent Application Publication
(10) Pub. No.: US 2012/0029505 A1
(43) Pub. Date: Feb. 2, 2012

(54) Self-Leveling Electrode Sets for Renal Nerve Ablation

(76) Inventor: Mark L. Jenson, Greenfield, MN (US)

(21) Appl. No.: 13/188,687

(22) Filed: Jul. 22, 2011

Related U.S. Application Data
(60) Provisional application No. 61/369,450, filed on Jul. 30, 2010.
(60) Provisional application No. 61/491,722, filed on May 31, 2011.

(51) Int. Cl. A61B 18/18

(52) U.S. Cl. 606/34; 606/41

(57) ABSTRACT

A catheter comprises a flexible shaft having a length for accessing the renal artery and a multiplicity of electrode sets each supported a support member. Each electrode set extends beyond the catheter's distal end and includes several elongated resilient members comprising a pre-formed curve and supporting an electrode. The resilient members are constrained to a low profile when encompassed by a wall of a removable sheath or a lumen wall of the catheter's shaft, and expand outwardly to assume a shape of their pre-formed curve when removed from the removable sheath or shaft lumen. The resilient members have a stiffness sufficient to maintain contact between the electrodes and an inner wall of the renal artery including irregularities of the inner wall of the renal artery. One or more temperature sensors can be situated at or proximate the plurality of electrode sets.

### RELATED APPLICATIONS

This application claims the benefit of Provisional Patent Application Ser. Nos. 61/369,450 filed Jul. 30, 2010 and 61/491,722 filed May 31, 2011, to which priority is claimed pursuant to 35 U.S.C. §119(e) and which are hereby incorporated herein by reference in their entirety.

### SUMMARY

Embodiments of the disclosure are generally directed to apparatuses and methods for ablating target tissue of the body from within a vessel. Embodiments are directed to high frequency AC ablation catheters, systems, and methods that employ self-leveling electrode sets for enhanced apposition of electrodes within a target vessel, particularly for irregularities along the inner wall of the vessel. Various embodiments of the disclosure are directed to apparatuses and methods for ablating perivascular renal nerves, such as for treatment of hypertension.

According to various embodiments, an apparatus includes a catheter comprising a flexible shaft having a proximal end, a distal end, and a length, the length of the shaft sufficient to access a target vessel, such as a patient's renal artery, relative to a percutaneous access location. The catheter includes a multiplicity of electrode sets each supported by one of a number of support members. The electrode sets are extendable beyond the distal end of the catheter when in a deployed configuration.

Each of the electrode sets includes a multiplicity of elongated resilient members comprising a pre-formed curve and supported by one of the support members. The resilient members are constrained to a low profile when encompassed by a wall of a removable sheath or a lumen wall of the catheter's shaft and, when removed from the removable sheath or lumen of the shaft, the resilient members expand outwardly and assume a shape of the pre-formed curve.

An electrode is provided at a distal end of each of the resilient members. The resilient members have a stiffness sufficient to maintain contact between the electrodes and an inner wall of the renal artery including irregularities of the inner wall of the renal artery. One or more temperature sensors can be situated at or proximate the plurality of electrode sets.

In various embodiments, each of the electrode sets is coupled to one of the support members, and each of the support members defines or is coupled to a respective conductor that extends along the length of the shaft and configured to couple to a control unit. The electrode sets are configured to deliver electrical current to the renal artery wall in accordance with an energy delivery protocol implemented by the control unit. The electrode sets can be configured to simultaneously or sequentially deliver electrical current to the renal artery wall in accordance with predefined energy delivery protocols implemented by the control unit.

According to some embodiments, a system includes an ablation catheter and a control unit electrically coupled to the ablation catheter. The ablation catheter includes a flexible shaft having a length sufficient to access a target vessel of the body, and a multiplicity of electrode sets extendable beyond the distal end of the catheter. Each of the electrode sets is supported by one of a multiplicity of support members and includes a multiplicity of elongated resilient members comprising a pre-formed curve and supported by one of the support members. The resilient members are constrained to a low profile when in a non-deployed configuration within the shaft or a delivery sheath and expand outwardly to assume a shape of the pre-formed curve when in a deployed configuration within the target vessel.

An electrode is provided at a distal end of each of the resilient members. The resilient members have a stiffness sufficient to maintain contact between the electrodes and an inner wall of the target vessel including irregularities of the inner wall of the target vessel. The control unit is electrically coupled to the multiplicity of electrode sets and configured to deliver electrical current through a wall of the target vessel to ablate perivascular renal nerves in accordance with an energy delivery protocol.

In accordance with other embodiments, a method involves constraining each of a plurality of electrode sets supported by one of a plurality of support members to a low profile configuration within a removable sheath or a lumen of a catheter shaft. The method involves moving the electrode sets free of the sheath or catheter shaft lumen within a target vessel to allow the electrode sets to assume a pre-formed shape and expand to contact an inner wall of the target vessel. The method further involves resiliently maintaining contact between one or more electrodes of each electrode set and an inner wall of the target vessel including irregularities of the inner wall of the target vessel, and ablating target tissue using the electrode sets.

These and other features can be understood in view of the following detailed discussion and the accompanying drawings.

### BRIEF DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery;

FIGS. 3B and 3C illustrate a portion of a renal nerve;

FIG. 4 illustrates an apparatus for ablating target tissue of a vessel of the body in accordance with various embodiments;

FIG. 5 illustrates a catheter which includes a multiplicity of electrode sets distributed at different circumferential locations at the distal end of the catheter's shaft in accordance with various embodiments;

FIG. 6 is a cross section of the shaft illustrated in FIG. 5;

FIG. 7 is an illustration of an electrode set which includes four electrodes supported by respective resilient members shown in a deployed configuration according to various embodiments;

FIGS. 8 and 9 are top views of the electrode set shown in FIG. 7 according to various embodiments;

FIGS. 10-12 illustrate electrode sets which include a number of temperature sensors in accordance with various embodiments;

FIGS. 13 and 14 illustrate integration of two or more temperature sensors within an electrode set in accordance with various embodiments; and

FIG. 15 shows a representative RF renal therapy apparatus in accordance with various embodiments.

### DISCLOSURE

Embodiments of the disclosure are directed to apparatuses and methods for ablating target tissue from within a vessel. Embodiments of the disclosure are directed to apparatuses and methods for ablating of perivascular renal nerves from within the renal artery for the treatment of hypertension. Embodiments of the disclosure include self-leveling electrode sets for delivering renal nerve ablation.

Maintaining good contact with the artery wall can be difficult with radiofrequency (RF) electrodes placed in the renal artery for ablation of perivascular renal nerves. This presents a particular problem in the irregular or diseased segments of the renal artery. There is need for improved electrode contact in renal artery based renal nerve ablation.

Embodiments of the disclosure are directed to apparatuses and methods for renal nerve ablation using electrodes contacting the renal artery wall. Apparatuses of the disclosure include a catheter with one or more sets of high frequency AC electrodes, such as RF electrodes. Within each electrode set, one or more electrodes are energized simultaneously. Each electrode is mounted on an elastic attachment such as a curved wire portion which can flex slightly when the electrode is pressed against the artery wall, but has sufficient stiffness to press against the wall with enough force to obtain good contact with the wall.

If the artery wall is curved or irregular, such as in a diseased or branching artery segment, one or more electrodes of the electrode set will maintain good electrical contact with the artery wall, even though other electrodes of the set may not. In this way, energizing the electrode set will produce effective RF heating in the perivascular tissues even in an irregularly-shaped artery.

Apparatuses of the disclosure can include additional sets of electrodes at separate axial and/or circumferential locations along the artery. The additional electrode sets can be energized in sequence, or all simultaneously, in unipolar or bipolar configurations, to effectively ablate at various locations along the artery to ablate the perivascular nerves.

One or more temperature sensors, such as thermocouples, may be provided at the site of each electrode set to measure the temperature of each electrode set. In some embodiments, a temperature sensor is positioned near or at the site of each electrode of the electrode set, allowing for precision temperature measurements at individual electrode locations of the ablation electrode arrangement.

Various embodiments of the disclosure are directed to apparatuses and methods for renal denervation for treating hypertension. Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

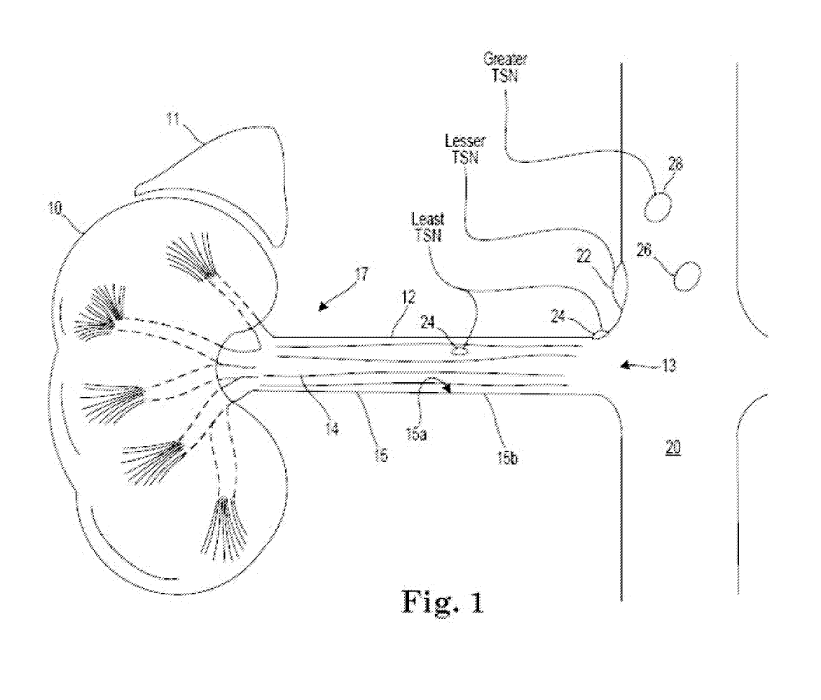

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the disclosure. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue. The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

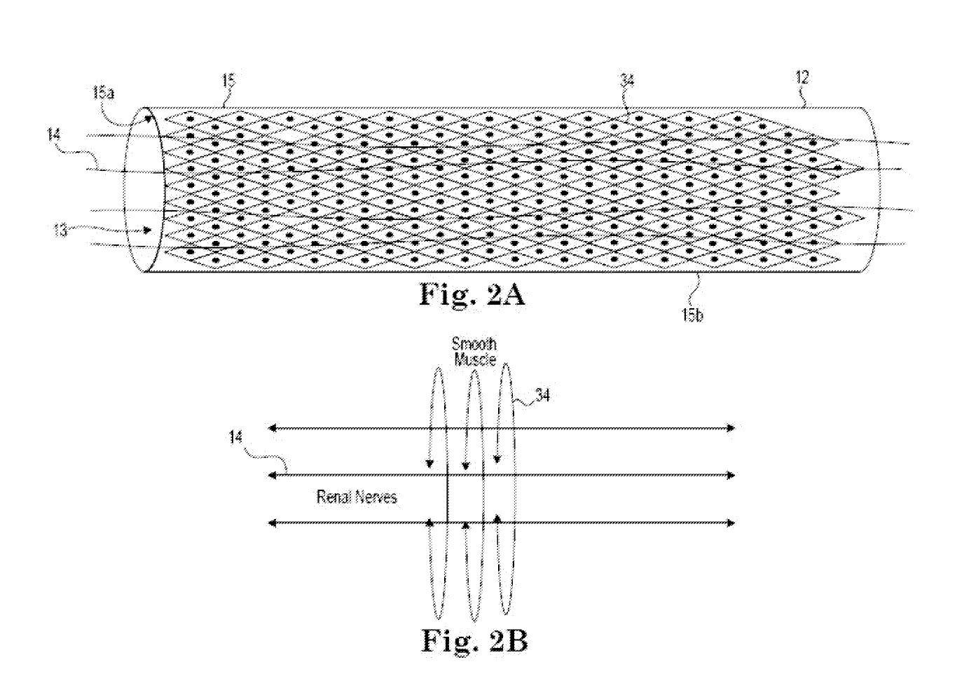

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

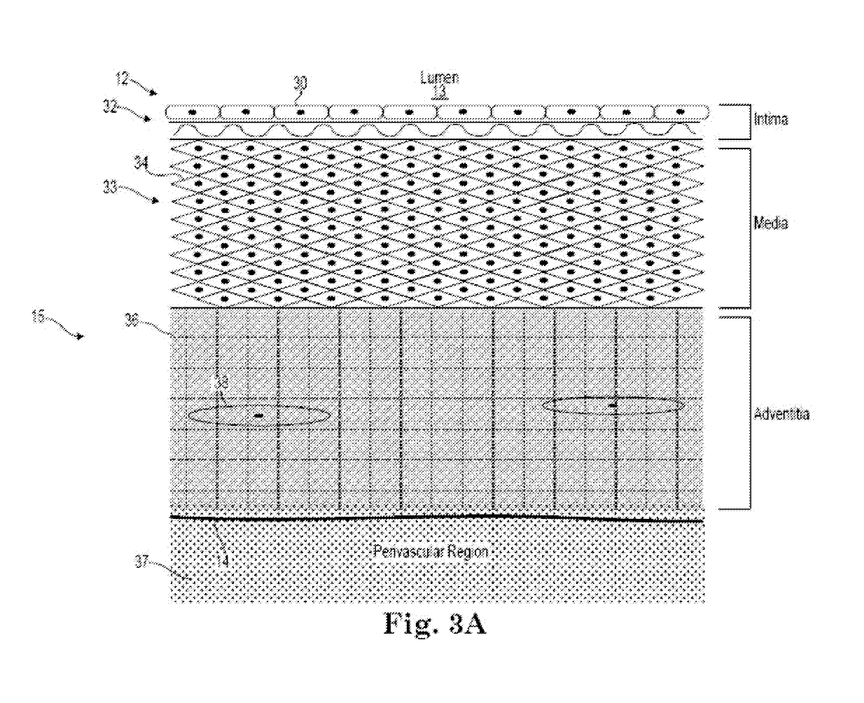

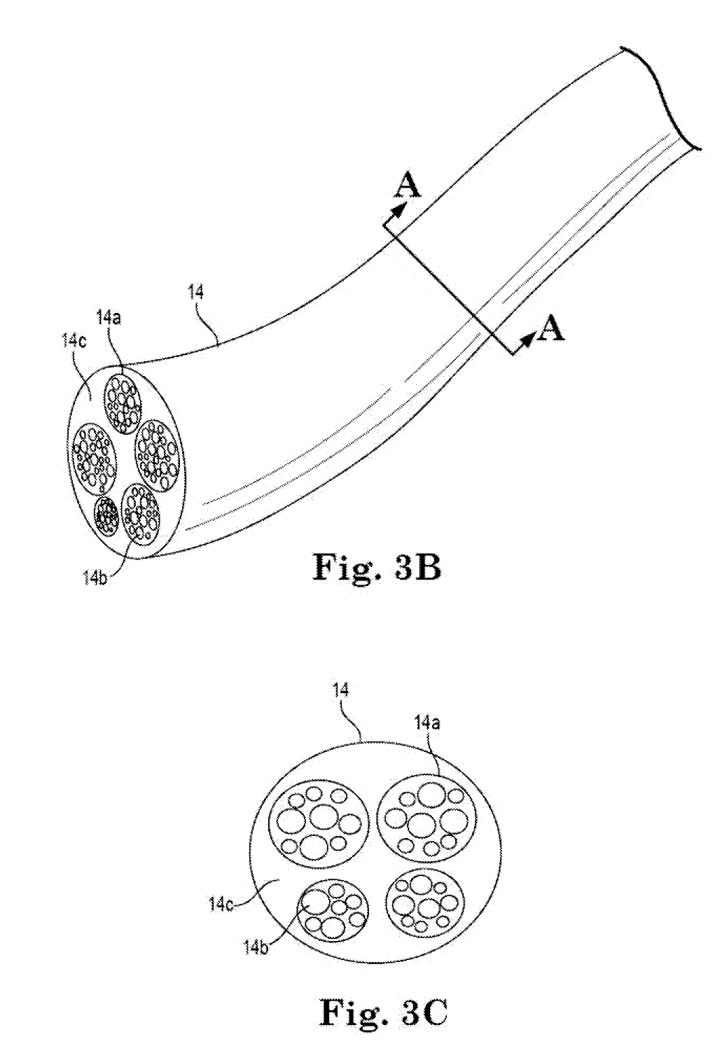

FIG. 3A shows a segment of a longitudinal cross-section through a renal artery, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing.

A perivascular region 37 is shown adjacent and peripheral to the adventitia 36 of the renal artery wall 15. A renal nerve 14 is shown proximate the adventitia 36 and passing through a portion of the perivascular region 37. The renal nerve 14 is shown extending substantially longitudinally along the outer wall 15 of the renal artery 12. The main trunk of the renal nerves 14 generally lies in or on the adventitia 36 of the renal artery 12, often passing through the perivascular region 37, with certain branches coursing into the media 33 to enervate the renal artery smooth muscle 34.

Embodiments of the disclosure may be implemented to provide varying degrees of denervation therapy to innervated renal vasculature. For example, embodiments of the disclosure may provide for control of the extent and relative permanency of renal nerve impulse transmission interruption achieved by denervation therapy delivered using a treatment apparatus of the disclosure. The extent and relative permanency of renal nerve injury may be tailored to achieve a desired reduction in sympathetic nerve activity (including a partial or complete block) and to achieve a desired degree of permanency (including temporary or irreversible injury).

Returning to FIGS. 3B and 3C, the portion of the renal nerve 14 shown in FIGS. 3B and 3C includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus.

Major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation.

In some embodiments, a treatment apparatus of the disclosure may be implemented to deliver denervation therapy that causes transient and reversible injury to renal nerve fibers 14b. In other embodiments, a treatment apparatus of the disclosure may be implemented to deliver denervation therapy that causes more severe injury to renal nerve fibers 14b, which may be reversible if the therapy is terminated in a timely manner. In preferred embodiments, a treatment apparatus of the disclosure may be implemented to deliver denervation therapy that causes severe and irreversible injury to renal nerve fibers 14b, resulting in permanent cessation of renal sympathetic nerve activity. For example, a treatment apparatus may be implemented to deliver a denervation therapy that disrupts nerve fiber morphology to a degree sufficient to physically separate the endoneurium tube of the nerve fiber 14b, which can prevent regeneration and re-innervation processes.

By way of example, and in accordance with Seddon's classification as is known in the art, a treatment apparatus of the disclosure may be implemented to deliver a denervation therapy that interrupts conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process in which the part of the axon separated from the neuron's cell nucleus degenerates. This process is also known as anterograde degeneration. Neurapraxia is the mildest form of nerve injury that may be imparted to renal nerve fibers 14b by use of a treatment apparatus according to embodiments of the disclosure.

A treatment apparatus may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b are preserved. Because axonal continuity is lost, Wallerian degeneration occurs. Recovery from axonotmesis occurs only through regeneration of the axons, a process requiring time on the order of several weeks or months. Electrically, the nerve fiber 14b shows rapid and complete degeneration. Regeneration and re-innervation may occur as long as the endoneural tubes are intact.

A treatment apparatus may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

The first and second degrees of nerve injury in the Sunderland system are analogous to Seddon's neurapraxia and axonotmesis, respectively. Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

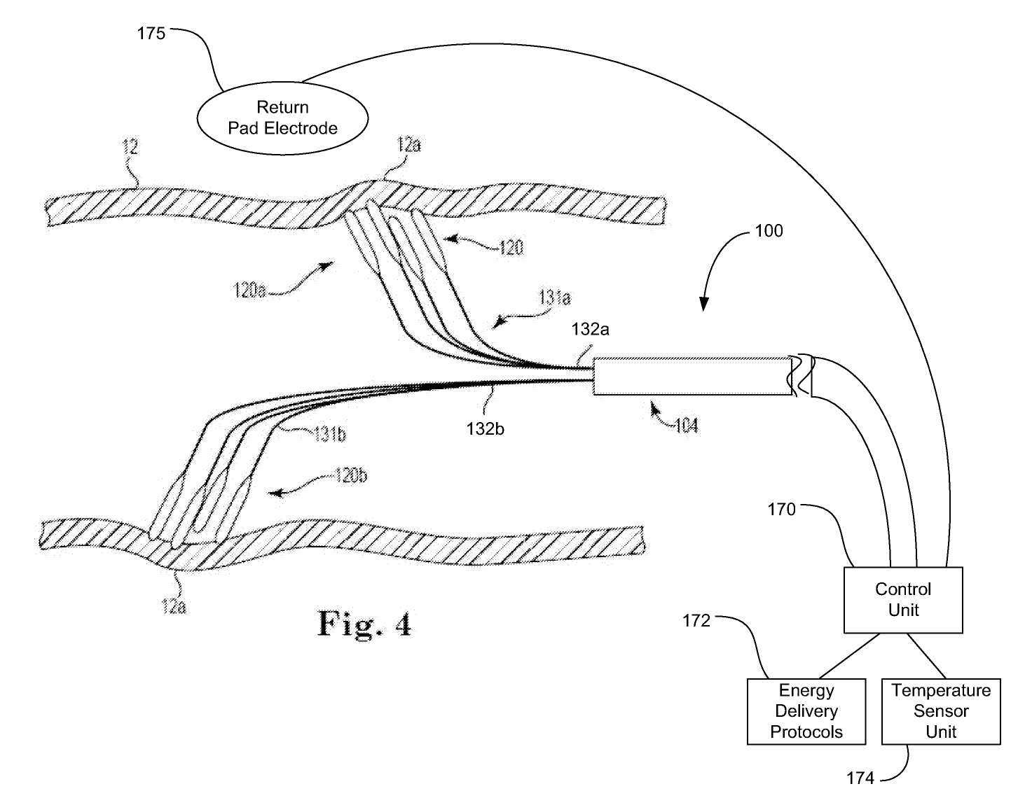

Turning now to FIG. 4, there is illustrated an apparatus for ablating target tissue of a vessel of the body in accordance with various embodiments. According to some embodiments, and as shown in FIG. 4, a catheter 100 includes a flexible shaft 104 having a proximal end, a distal end, and a length. The length of the shaft is sufficient to access a target vessel of the body, such as a patient's renal artery 12, relative to a percutaneous access location.

The catheter shaft 104 includes a number of lumens through which a multiplicity of sets 120a, 120b of electrodes 120 extend. Each of the electrode sets 120a, 120b is supported by a respective support member 132a, 132b, such that the electrode sets 120a, 120b extend beyond the distal end of the catheter shaft 104 in a deployed configuration. In FIG. 4, two electrode sets 102a, 120b are shown for purposes of explanation. It is understood that one or more than two electrode sets may be provided in various embodiments to treat a predefined arc of a target vessel (e.g., 90° or 180°) or 360° or more of the target vessel's circumference. For example, between three and six electrode sets may be provide so that at least one full revolution of a target vessel's wall can be treated without having to reposition the catheter shaft 104 to complete the ablation procedure.

In some embodiments, the support members 132a, 132b are fixed at the catheter's distal end. In other embodiments, the support members 132a, 132b are displaceable within their respective lumens of the shaft 104. In such embodiments, the electrode sets 120a, 120b can be retracted into the body of the shaft 104, such as for purposes of catheter delivery into and extraction from the renal artery 12, and extended beyond the distal end of the shaft 104, such as for delivering denervation therapy to the renal artery 12.

As is further shown in FIG. 4, each of the electrode sets 120a, 120b comprises a multiplicity of elongated resilient members 131a, 131b comprising a pre-formed curve and supported by one of the support members 132a, 132b. The resilient members 131a, 131b are constructed to be collapsible when encompassed by a wall of a removable sheath or lumen wall of the shaft 104, and expand outwardly when removed from the removable sheath or extended from the shaft lumen.

The resilient members 131a, 131b are preferably constructed as a single or a multiple element structure, providing high or superelastic properties and good electrical conduction properties. For example, the resilient members 131a, 131b can be constructed to have a shape memory, such that the resilient members 131a, 131b expand outwardly and assume a shape of the pre-formed curve when in a deployed configuration. In some embodiments, the resilient members 131a, 131b are constructed to assume different shapes, such that at least some of the resilient members 131a, 131b expand both longitudinally and circumferentially to assume the shape of their respective pre-formed curves.

For example, a distal region of the resilient members 131a, 131b of each electrode set 120a, 120b, including their respective electrodes 120, can take on a longitudinally spaced configuration when deployed. By way of further example, a distal region of the resilient members 131a, 131b of each electrode set 120a, 120b, including their respective electrodes 120, can take on a longitudinally spaced and circumferentially offset configuration when deployed.

An electrode 120 is provided at a distal end of each of the resilient members 131a, 131b. The resilient members 131a, 131b preferably have a stiffness sufficient to maintain contact between the electrodes 120 and an inner wall of the renal artery 12 including irregularities 12a of the renal artery's inner wall.

According to various embodiments, each of the resilient members 131a, 131b is constructed from an electrically conductive material and configured as a wire. Each of the conductive resilient members 131a, 131b of an electrode set 120a, 120b is coupled to a support member 132a, 132b, which is preferably constructed as a conductive wire. The resilient members 131a, 131b may be constructed from a shape memory alloy, such as Nitinol.

As is shown in FIG. 4, the resilient members 131a, 131b of each electrode set 120a, 120b are preferably physically and electrically conjoined at one of the multiplicity of support members 132a, 132b. In some embodiments, the resilient members 131a, 131b of each electrode set 120a, 120b can be welded at a distal end of a respective support member 132a, 132b, such as at a common location or at separate locations of the support members 132a, 132b. In other embodiments, the resilient members 131a, 131b of each electrode set 120a, 120b can be formed from a common support wire using various known extrusion or cutting techniques. The support members 132a, 132b can extend along the length of the shaft 104 or couple to conductors that extend along the length of the shaft 104. Support members 132 can include both a structural spring-like element (such as elastic or superelastic nitinol or a spring-like stainless steel) and a superior electrical conductor (such as stainless steel or platinum), or a single element can provide both the spring-like support and the electrical conduction properties.

In accordance with various embodiments, each of the electrode sets 120a, 120b is coupled to one of the support members 132a, 132b, and the support members 132a, 132b are either configured as, or coupled to, respective conductors that extend along the length of the shaft and are configured to couple to a control unit 170. The control unit 170 includes an RF generator that can be controlled to deliver different RF therapies according to various predefined energy delivery protocols 172.

In some approaches, the electrode sets 120a, 120b are configured to simultaneously deliver electrical current to the renal artery wall in accordance with a predefined energy delivery protocol implemented by the control unit 170. In other approaches, the electrode sets 120a, 120b are configured to sequentially deliver electrical current to the renal artery wall in accordance with a predefined energy delivery protocol implemented by the control unit 170. A unipolar energy delivery configuration can be employed by use of a return pad electrode 175. A bipolar energy delivery configuration can be implemented by selectively activating combinations of the electrode sets 120a, 120b.

In some embodiments, as discussed below, the control unit 170 may include a temperature sensor unit 174 that receives signals from one or more temperature sensors situated at or near the electrodes 120. The RF generator can be automatically controlled based on temperature at the electrode-tissue interface as indicated by the temperature sensor unit 174.

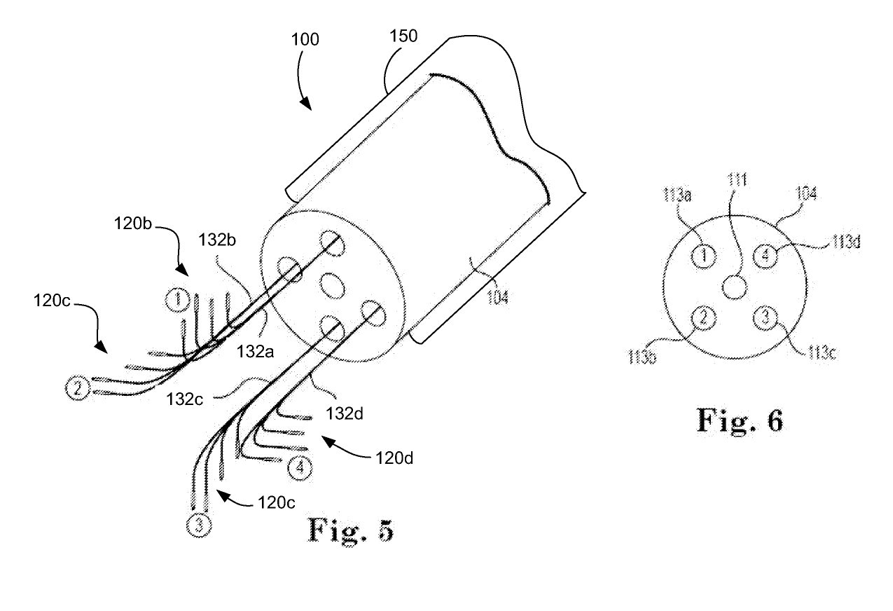

FIG. 5 illustrates a catheter 100 which includes a multiplicity of electrode sets distributed at different circumferential locations at the distal end of the shaft 104 of catheter 100 in accordance with various embodiments. FIG. 6 shows a cross section of the shaft 104 illustrated in FIG. 5. In FIG. 5, four electrode sets 120a-120d are shown. The electrode sets 120a-120d are shown distributed in a spaced-apart relationship at a separation of about 90° from one another. In this configuration, the electrode sets 120a-120d can deliver ablation therapy to a full revolution of the renal artery wall without having to reposition the catheter shaft 104 during the ablation procedure. The electrode sets 120a-120d can also be spaced apart longitudinally to deliver the full circumferential ablation therapy but at different axial positions to minimize arterial injury at any particular axial location without having to reposition the catheter. Although four electrode sets 120a-120d are shown in FIG. 5, it is understood that more or fewer than four electrode sets can be used to supply an amount of energy sufficient to ablate perivascular renal nerves over a full revolution of the renal artery wall.

In FIGS. 5 and 6, the shaft 104 is shown to include a guide lumen 111 dimensioned to receive a guidewire or other elongated navigation assist member that can by used by the clinician to facilitate delivery of the catheter's distal end into a desired treatment location, such as a renal artery. In the configuration shown in FIGS. 5 and 6, the guidewire lumen 111 defines an open lumen of the shaft 104, which allows for advancement of a guidewire therethrough for navigating the distal end of the catheter 100 to the renal artery, for example. After the guidewire is positioned within the renal artery, the catheter 100 can be advanced along the guidewire and delivered to the lumen of the renal artery using an over-the-wire delivery technique.

In embodiments where the electrode sets 120a-120d can retract into their respective lumens 113a-113d of the shaft 104 during delivery and extraction, the resilient members 131 supporting the electrodes of the electrode sets 120a-120d are constrained to a low profile while encompassed by their respective lumens 113a-113d or by a delivery sheath according to some embodiments. In embodiments where the electrode sets 120a-120d are fixedly positioned at the distal tip of the catheter shaft 104, a delivery sheath 150 can be advanced over the guidewire and into the destination vessel, and the catheter 100 can be advanced through the delivery sheath 150, with the resilient members supporting the electrodes of the electrode sets 120a-120d being constrained to a low profile while encompassed by the delivery sheath.

FIG. 7 is an illustration of an electrode set 120a which includes four electrodes 120 supported by respective resilient members 131 shown in a deployed configuration. In FIG. 7, each resilient member 131 has a lateral height, h₁-h₄, relative to a longitudinal axis of a support member 132 (shown at height h₀) to which the four resilient members 131 are conjoined. The lateral height of each electrodes 120, therefore, varies in accordance with the lateral height of its respective resilient member 131. The variation in height among the electrodes 120 of the electrode set 120a advantageously accommodates irregularities 12a of the inner wall of the renal artery 12. The variation in height among the electrodes 120 of the electrode set 120a also provides for continuous electrode-to-tissue contact by at least one of the electrodes 120 of the electrode set 120a and the inner wall of the renal artery 12, even in regions of the renal artery having inner wall irregularities 12a. In some embodiments, the separate resilient members 131 urge individual electrodes 120 in each electrode set to contact the renal artery 12, so that most or all of the electrodes 120 are in contact with the wall of the renal artery 12.

FIG. 7 also shows an inter-electrode spacing between adjacent electrodes 120. This inter-electrode spacing can be the same or differ for adjacent electrodes 120. For example, an inter-electrode spacing, d₁-d₂, between electrodes 1 and 2 shown in FIG. 7 can be the same as, or differ from, an inter-electrode spacing, d₂-d₃, between electrodes 2 and 3. Inter-electrode spacing can be adjusted based on relative height differences between adjacent electrodes, for example.

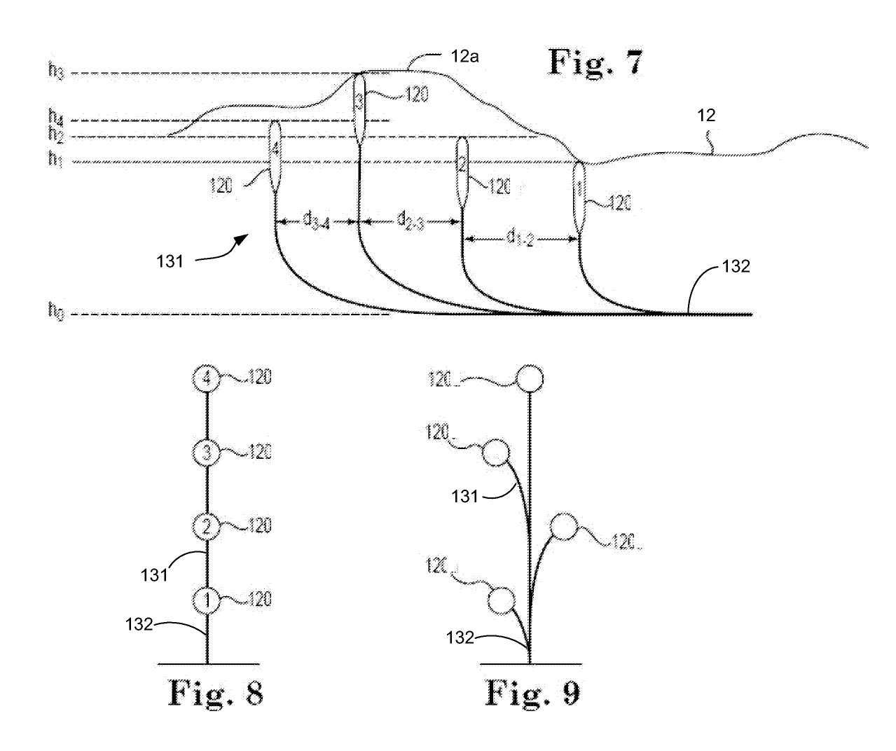

FIGS. 8 and 9 are top views of the electrode set 120a shown in FIG. 7 according to various embodiments. In the embodiment shown in FIG. 8, the resilient members 131 supporting each of the electrodes 120 are arranged with their longitudinal axes in alignment with a longitudinal axis of the support member 132 to which each resilient member 131 is connected. In the embodiment shown in FIG. 9, some of the resilient members 131 supporting the electrodes 120 are arranged with their longitudinal axes laterally offset relative to the longitudinal axis of the support member 132 to which the resilient members 131 are connected. The degree of lateral axial offset may be the same or differ for each resilient member 131, as can the inter-electrode spacing.

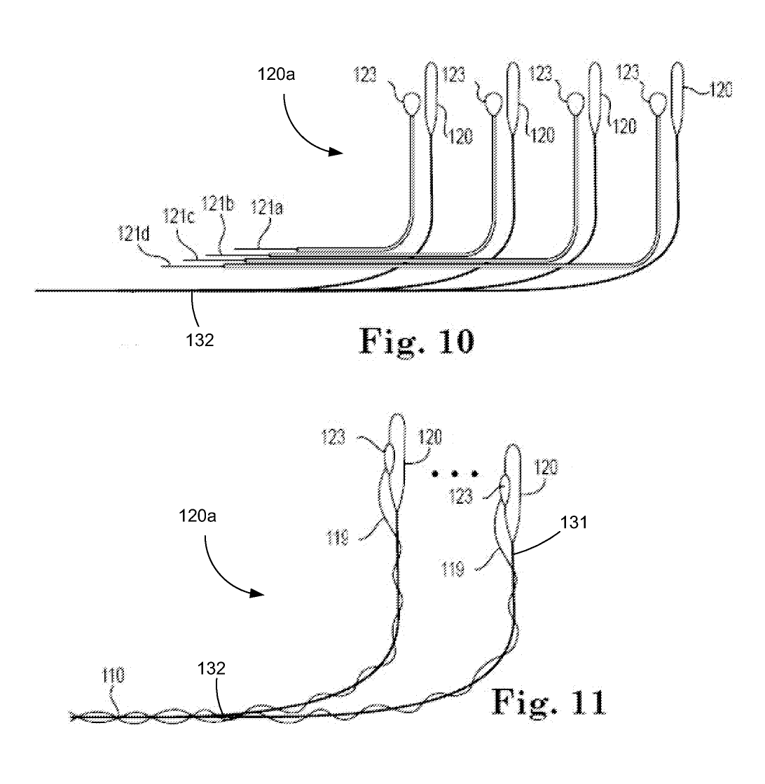

FIG. 10 illustrates an electrode set 120a which includes a number of temperature sensors, such as thermocouples, in accordance with various embodiments. In the embodiment shown in FIG. 10, one of a number of temperature sensors 123a-123d is thermally associated with one of a multiplicity of electrodes 120. The temperature sensors 123a-123d are coupled to a respective sensor wire 121a-121d, each of which extends along the length of the shaft 104 (not shown) of the catheter 100. The sensor wires 121a-121d are shown in FIG. 10 to include an electrically insulating sleeve or coating.

The temperature sensors 123a-123d shown in FIG. 10 define an array structure separate from that of the electrode set 120a. The sensor wires 121a-121d, for example, can be formed from a superelastic or shape memory alloy and bundled to a common clinician-manipulatable wire so that the array configuration of the temperature sensors 123a-123d is assumed when deployed in the renal artery. In this configuration, the temperature sensor array 123a-123d can be moved by the clinician, both longitudinally and circumferentially, to sense temperatures at or nearby the electrode-tissue interface. The lateral height of the temperature sensors 123a-123d can vary from that shown in FIG. 10. For example, the lateral height of each temperature sensor 123a-123d can be the same as that of its associated electrode 120.

FIG. 11 illustrates an electrode set 120a which includes a number of temperature sensors, such as thermocouples, in accordance with various embodiments. In the embodiment shown in FIG. 11, one of a number of temperature sensors 123a-123n is coupled to one of a multiplicity of electrodes 120 of the electrode set 120a. The temperature sensors 123a-123n can be bonded to their respective electrode 120 using a thermally conductive adhesive, for example. A sensor wire 119a-119n is connected to each of the sensors 123a-123n and runs along the length its respective resilient member 131.

In the configuration shown in FIG. 11, the sensor wires 119a-119n are pliable insulated wires that can wrap around the resilient members 131 in a barber pole manner. In other configurations, the sensor wires 119a-119n can run parallel, and be bonded, to the resilient members 131. In further embodiments, the resilient members can be covered with an electrically insulating sleeve or coating, in which case the sensor wires 119a-119n need not have an electrically insulating sleeve or coating.

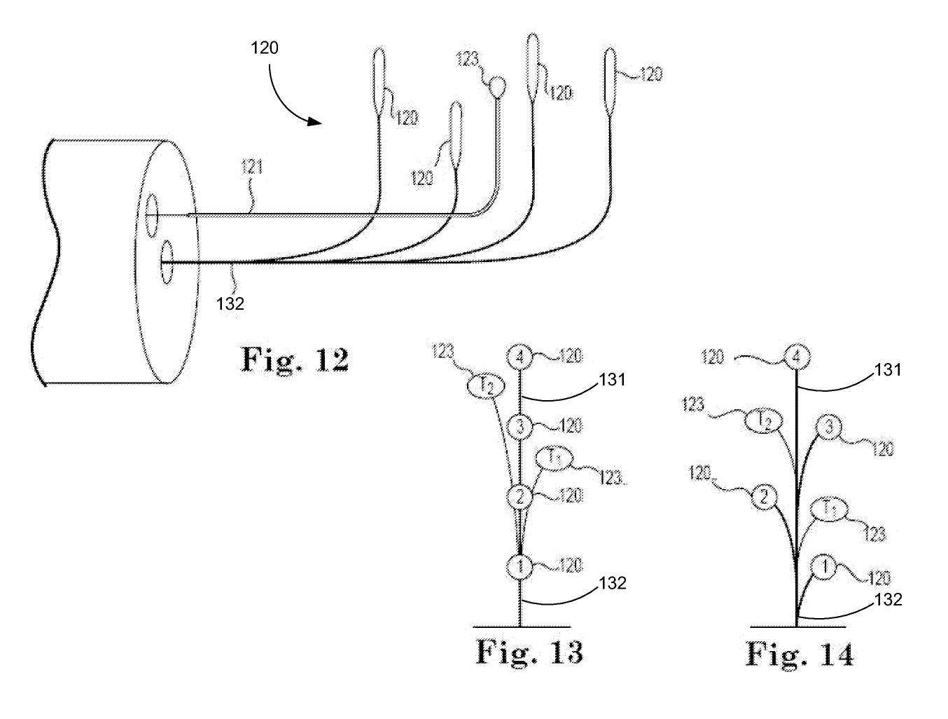

FIG. 12 shows an electrode set 120a which includes a single temperature sensor 123, such as thermocouples, in accordance with various embodiments. In the embodiment shown in FIG. 12, a temperature sensor 123 is supported by a resilient sensor wire 121, which is shown to include an electrically insulating sleeve or coating. The temperature sensor wire 121 may be configured as, or coupled to, a more robust wire (e.g., like a guidewire) that facilitates displacement and rotation of the temperature sensor 123 by a clinician. The temperature sensor wire is shown extending from a sensor lumen of the shaft 104, which is a lumen separate from the lumen dimensioned to receive the electrode support/conductor 132. A single temperature sensor embodiment has the advantage of reduced complexity, yet provides a useful indication of electrode-tissue interface temperature for the electrode set 120a during the ablation procedure.

FIGS. 13 and 14 illustrate integration of two or more temperature sensors within an electrode set 120a in accordance with various embodiments. In the embodiment shown in FIG. 13, two or more temperature sensors 123 can be supported by resilient members that are axially offset somewhat from a longitudinal axis of the axially aligned resilient members 131 supporting the electrodes 120. In FIG. 14, some of the resilient members 131 supporting the electrodes 120 are arranged with their longitudinal axes laterally offset relative to the longitudinal axis of the support member 132 to which the resilient members 131 are connected. Two or more temperature sensors 123 can be supported by resilient members that are laterally offset somewhat from a longitudinal axis of the support member 132.

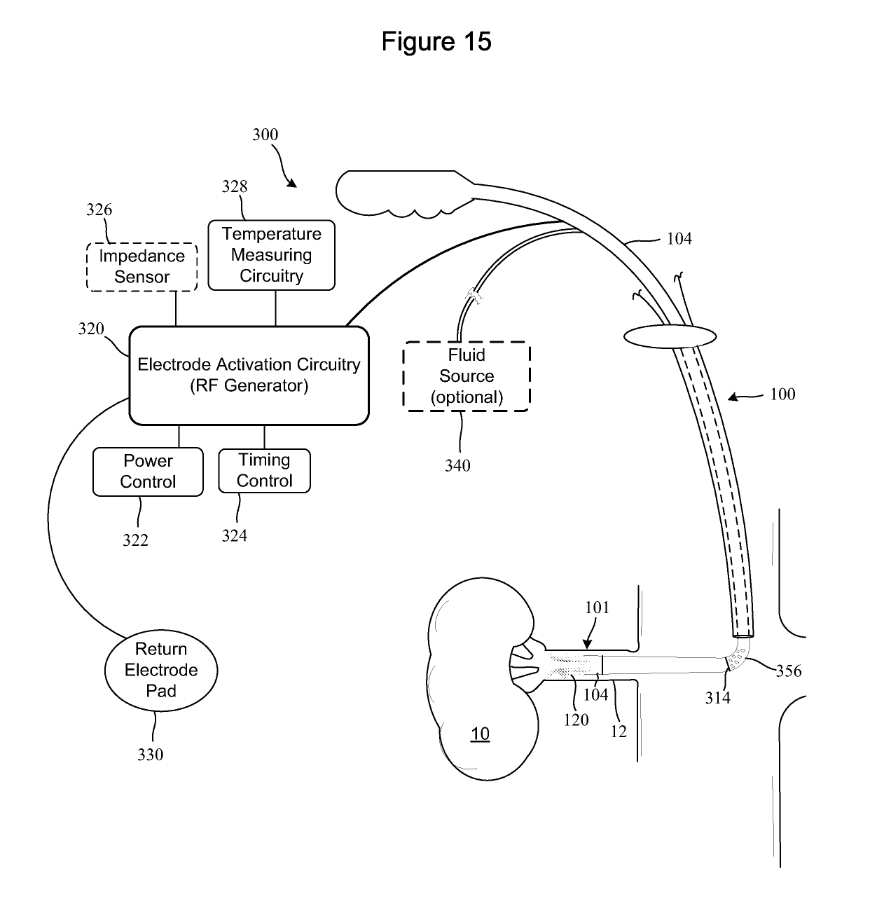

FIG. 15 shows a representative RF renal therapy apparatus 300 in accordance with various embodiments of the disclosure. The apparatus 300 illustrated in FIG. 15 includes external electrode activation circuitry 320 which comprises power control circuitry 322 and timing control circuitry 324. The external electrode activation circuitry 320, which includes an RF generator, is coupled to temperature measuring circuitry 328 and may be coupled to an optional impedance sensor 326. The catheter 100 includes a shaft 104 that incorporates a lumen arrangement 105 configured for receiving a variety of components, such as conductors, pharmacological agents, actuator elements, obturators, sensors, or other components as needed or desired.

The RF generator of the external electrode activation circuitry 320 may include a return pad electrode 330 that is configured to comfortably engage the patient's back or other portion of the body near the kidneys. Radiofrequency energy produced by the RF generator is coupled to the treatment element 101 at the distal end of the catheter 101 by the conductor arrangement 110 disposed in the lumen of the catheter's shaft 104.

Renal denervation therapy using the apparatus shown in FIG. 15 is typically performed using one or more electrode sets 119 of the treatment element 101 positioned within the renal artery 12 and the return pad electrode 330 positioned on the patient's back, with the RF generator operating in a monopolar mode. In this implementation, the electrode sets 120a-120n are configured for operation in a unipolar configuration. In other implementations, the electrodes 120 of the one or more electrode sets 120a-120n can be configured for operation in a bipolar configuration, in which case the return electrode pad 330 is not needed. The radiofrequency energy flows through the one or more electrode sets 120a-120n in accordance with a predetermined activation sequence (e.g., sequential or concurrent) and ablates target tissue which includes renal nerves.

In general, when renal artery tissue temperatures rise above about 113° F. (50° C.), protein is permanently damaged (including those of renal nerve fibers). For example, any mammalian tissue that is heated above about 50° C. for even 1 second is killed. If heated over about 65° C., collagen denatures and tissue shrinks. If heated over about 65° C. and up to 100° C., cell walls break and oil separates from water. Above about 100° C., tissue desiccates.

According to some embodiments, the electrode activation circuitry 320 is configured to control activation and deactivation of the electrode sets 120a-120n in accordance with a predetermined energy delivery protocol and in response to signals received from temperature measuring circuitry 328. The electrode activation circuitry 320 controls radiofrequency energy delivered to the electrode sets 120a-120n so as to maintain the current densities at a level sufficient to cause heating of the target tissue to at least a temperature of 55° C.

Temperature sensors 123 situated at the treatment element 101 provide for continuous monitoring of renal artery tissue temperatures, and RF generator power is automatically adjusted so that the target temperatures are achieved and maintained. An impedance sensor arrangement 326 may be used to measure and monitor electrical impedance during RF denervation therapy, and the power and timing of the RF generator 320 may be moderated based on the impedance measurements or a combination of impedance and temperature measurements. The size of the ablated area is determined largely by the size, number, and shape of the electrodes of the electrode sets 120a-120n at the treatment element 101, the power applied, and the duration of time the energy is applied.

Marker bands 314 can be placed on one or multiple parts of the treatment element 101 to enable visualization during the procedure. Other portions of the catheter 101, such as one or more portions of the shaft 104 (e.g., at hinge mechanism 356), may include a marker band 314. The marker bands 314 may be solid or split bands of platinum or other radiopaque metal, for example. Radiopaque materials are understood to be materials capable of producing a relatively bright image on a fluoroscopy screen or another imaging technique during a medical procedure. This relatively bright image aids the user in determining specific portions of the catheter 100, such as the tip of the catheter 101, the treatment element 101, and the hinge 356, for example. A braid and/or electrodes of the catheter 100, according to some embodiments, can be radiopaque.

It is to be understood that even though numerous characteristics of various embodiments have been set forth in the foregoing description, together with details of the structure and function of various embodiments, this detailed description is illustrative only, and changes may be made in detail, especially in matters of structure and arrangements of parts illustrated by the various embodiments to the full extent indicated by the broad general meaning of the terms in which the appended claims are expressed.

What is claimed is:

1. An apparatus, comprising:
a catheter comprising a flexible shaft having a proximal end, a distal end, and a length, the length of the shaft sufficient to access a patient's renal artery relative to a percutaneous access location; and
a plurality of electrode sets extendable beyond the distal end of the catheter, each of the electrode sets supported by one of a plurality of support members and comprising:
a plurality of elongated resilient members comprising a pre-formed curve and supported by one of the support members, the resilient members constrained to a low profile when encompassed by a wall of a removable sheath or a lumen wall of the shaft and, when removed from the removable sheath or lumen of the shaft, the resilient members expanding outwardly and assuming a shape of the pre-formed curve; and
an electrode provided at a distal end of each of the resilient members, the resilient members having a stiffness sufficient to maintain contact between the electrodes and an inner wall of the renal artery including irregularities of the inner wall of the renal artery.

2. The apparatus according to claim 1, wherein each of the resilient members comprises an electrically conductive wire constructed from a highly elastic or superelastic material.

3. The apparatus according to claim 1, wherein the resilient members of each electrode set are physically and electrically conjoined at one of the plurality of support members, the support members defining or coupled to respective conductors that extend along the length of the shaft.

4. The apparatus according to claim 1, wherein the shaft comprises a plurality of lumens, each of the plurality of lumens dimensioned to constrain at least one of the plurality of electrode sets to a low profile when encompassed by the lumens.

5. The apparatus according to claim 1, wherein a distal region of the resilient members of each electrode set including the respective electrodes take on a longitudinally spaced configuration.

6. The apparatus according to claim 1, wherein a distal region of the resilient members of each electrode set including the respective electrodes take on a longitudinally spaced and circumferentially offset configuration.

7. The apparatus according to claim 1, wherein the catheter shaft comprises a lumen dimensioned to receive a guidewire.

8. The apparatus according to claim 1, comprising one or more temperature sensors situated at or proximate the plurality of electrode sets.

9. The apparatus according to claim 1, comprising a temperature sensors situated at or proximate each electrode of each of the plurality of electrode sets.

10. The apparatus according to claim 1, comprising:
one or more temperature sensors situated at or proximate the plurality of electrode sets;
wherein the one or more temperature sensors are supported by a wire arrangement extending along the length of the shaft, the wire arrangement facilitating clinician displacement and rotation of the one or more temperatures sensors within the renal artery.

11. A system, comprising:
an ablation catheter, comprising:
a flexible shaft having a length sufficient to access a target vessel of the body; and
a plurality of electrode sets extendable beyond the distal end of the catheter, each of the electrode sets supported by one of a plurality of support members and comprising:
a plurality of elongated resilient members comprising a pre-formed curve and supported by one of the support members, the resilient members constrained to a low profile when in a non-deployed configuration within the shaft or a delivery sheath and expanding outwardly and assuming a shape of the pre-formed curve when in a deployed configuration within the target vessel; and
an electrode provided at a distal end of each of the resilient members, the resilient members having a stiffness sufficient to maintain contact between the electrodes and an inner wall of the target vessel including irregularities of the inner wall of the target vessel; and
a control unit electrically coupled to the plurality of electrode sets and configured to deliver electrical current through a wall of the target vessel to ablate target tissue in accordance with an energy delivery protocol.

12. The system of claim 11, wherein the electrode sets are configured to simultaneously deliver electrical current to the vessel wall in accordance with the energy delivery protocol implemented by the control unit.

13. The apparatus according to claim 11, wherein the electrode sets are configured to sequentially deliver electrical current to the vessel wall in accordance with the energy delivery protocol implemented by the control unit.

14. The apparatus according to claim 11, comprising:
one or more temperature sensors situated at or proximate the plurality of electrode sets;
wherein the control unit is electrically coupled to the one or more temperature sensors and configured to deliver electrical current through the wall of the target vessel to ablate the target tissue in accordance with an energy delivery protocol and in response to vessel wall temperature measured by the one or more temperature sensors.

15. The apparatus according to claim 14, wherein at least one temperature sensor is situated at or proximate each electrode of each of the plurality of electrode sets.

16. The apparatus according to claim 11, wherein each of the resilient members comprises an electrically conductive wire constructed from a highly elastic or superelastic material.

17. The apparatus according to claim 11, wherein the resilient members of each electrode set are physically and electrically conjoined at one of the plurality of support members, the support members defining or coupled to respective conductors that extend along the length of the shaft and electrically couple with the control unit.

18. A method, comprising:
constraining each of a plurality of electrode sets supported by one of a plurality of support members to a low profile configuration within a removable sheath or a lumen of a catheter shaft;
moving the electrode sets free of the sheath or catheter shaft lumen within a target vessel to allow the electrode sets to assume a pre-formed shape and expand to contact an inner wall of the target vessel;
resiliently maintaining contact between one or more electrodes of each electrode set and an inner wall of the target vessel including irregularities of the inner wall of the target vessel; and
ablating target tissue using the electrode sets.

19. The method of claim 18, comprising measuring temperature at or proximate the one or more electrodes, and moderating target tissue ablation in response to temperature measurements.

20. The method of claim 18, comprising:
after completing ablation, constraining the electrode sets to the low profile configuration within the removable sheath or the lumen of the catheter shaft; and
removing the electrode sets from the target vessel while in the low profile configuration.

21. The method of claim 18, wherein the target vessel comprises a renal artery and the target tissue comprises perivascular renal nerves.
